(19) United States
(12) Patent Application Publication
(10) Pub. No.: US 2026/0260055 A1
(43) Pub. Date: Sep. 3, 2026

(54) CREATION DEVICE, CREATION METHOD, AND COMPUTER-READABLE MEDIUM

(71) Applicants: NEC Corporation, Tokyo (JP); SOCIAL WELFARE ORGANIZATION SAISEIKAI IMPERIAL GIFT FOUNDATION, INC. SAISEIKAI KUMAMOTO HOSPITAL, Kumamoto-shi (JP)

(72) Inventors: Shuhei NOYORI, Tokyo (JP); Kei SHIBUYA, Tokyo (JP); Konosuke TEMMEI, Tokyo (JP); Mieko MANO, Tokyo (JP); Keiichirou OKAMOTO, Tokyo (JP); Toshiharu SASSA, Kumamoto (JP); Miho Hashimoto, Kumamoto (JP); Chie ONOUE, Kumamoto (JP); Yukio KOZUMA, Kumamoto (JP)

(73) Assignees: NEC Corporation, Tokyo (JP); SOCIAL WELFARE ORGANIZATION SAISEIKAI IMPERIAL GIFT FOUNDATION, INC. SAISEIKAI KUMAMOTO HOSPITAL, Kumamoto-shi (JP)

(21) Appl. No.: 19/553,538

(22) Filed: Mar. 2, 2026

(30) Foreign Application Priority Data
Mar. 3, 2025 (JP) 2025-032947

(51) Int. Cl. G06F 40/166; G06F 40/103; G16H 10/60; G16H 50/30

(52) U.S. Cl. CPC G06F 40/166; G06F 40/103; G16H 10/60; G16H 50/30

(57) ABSTRACT

A creation device according to the present disclosure includes a condition evaluation unit that evaluates a specific health condition of an individual using information recorded in a medical/health information database as an input, a variable importance evaluation unit that calculates a variable importance obtained by quantifying a magnitude of contribution to evaluation of the health condition for each variable using information including one or more variables used for the evaluation of the health condition as an input, a variable selection unit that determines whether each variable is included in a summary sentence using the variable importance as an input, and an instruction creation unit that creates a summary sentence creation instruction for creating a summary sentence summarizing information regarding the health condition using a variable determined to be included in the summary sentence as an input.

### INCORPORATION BY REFERENCE

This application is based upon and claims the benefit of priority from Japanese patent application No. 2025-032947, filed on Mar. 3, 2025, the disclosure of which is incorporated herein in its entirety by reference.

### TECHNICAL FIELD

The present disclosure relates to a creation device, a creation method, and a program.

### BACKGROUND ART

JP 2020-38602 A discloses an information generation device capable of generating appropriate summary information from medical record information. The information generation device acquires importance of each element by performing text analysis on the medical record document, and generates a summary document based on the importance.

### SUMMARY

Generally important elements in medical record sentences are not always useful for evaluating the condition of a specific individual. Therefore, there is a possibility that the technology described in JP 2020-38602 A cannot generate appropriate summary information for a specific individual.

The present disclosure has been made to solve such a problem, and an example object thereof is to provide a creation device, a creation method, and a program that enable creation of appropriate summary information for an individual.

A creation device according to an example aspect of the present disclosure includes
a condition evaluation unit that evaluates a specific health condition of an individual using information recorded in a medical/health information database as an input,
a variable importance evaluation unit that calculates a variable importance obtained by quantifying a magnitude of contribution to evaluation of the health condition for each variable using information including one or more variables used for the evaluation of the health condition as an input,
a variable selection unit that determines whether each variable is included in a summary sentence using the variable importance as an input, and
an instruction creation unit that creates a summary sentence creation instruction for creating a summary sentence summarizing information regarding the health condition using a variable determined to be included in the summary sentence as an input.

A creation method according to an example aspect of the present disclosure includes
evaluating a specific health condition of an individual using information recorded in a medical/health information database as an input,
calculating a variable importance obtained by quantifying a magnitude of contribution to evaluation of the health condition for each variable using information including one or more variables used for the evaluation of the health condition as an input,
determining whether each variable is included in a summary sentence using the variable importance as an input, and
creating a summary sentence creation instruction for creating a summary sentence summarizing information regarding the health condition using a variable determined to be included in the summary sentence as an input.

A program according to an example aspect of the present disclosure causes a computer to execute
a process of evaluating a specific health condition of an individual using information recorded in a medical/health information database as an input,
a process of calculating a variable importance obtained by quantifying a magnitude of contribution to evaluation of the health condition for each variable using information including one or more variables used for the evaluation of the health condition as an input,
a process of determining whether each variable is included in a summary sentence using the variable importance as an input, and
a process of creating a summary sentence creation instruction for creating a summary sentence summarizing information regarding the health condition using a variable determined to be included in the summary sentence as an input.

According to the present disclosure, it is possible to provide a creation device, a creation method, and a program that enable creation of appropriate summary information for an individual.

### BRIEF DESCRIPTION OF THE DRAWINGS

FIG. 1 is a block diagram illustrating a creation device according to the present disclosure;

FIG. 2 is a flowchart illustrating a creation method according to the present disclosure;

FIG. 3 is a block diagram illustrating a creation device according to the present disclosure;

FIG. 4 is a block diagram illustrating a creation device according to the present disclosure;

FIG. 5 is a block diagram illustrating a system configuration of the creation device according to the present disclosure;

FIG. 6 is a schematic view illustrating a display screen according to the present disclosure;

FIG. 7 is a schematic view illustrating a display screen according to the present disclosure;

FIG. 8 is a schematic view illustrating a display screen according to the present disclosure;

FIG. 9 is a schematic view illustrating a display screen according to the present disclosure;

FIG. 10 is a schematic view illustrating a display screen according to the present disclosure;

FIG. 11 is a schematic view illustrating a display screen according to the present disclosure;

FIG. 12 is a block diagram illustrating a creation device according to the present disclosure;

FIG. 13 is a schematic view illustrating a display screen according to the present disclosure;

FIG. 14 is a schematic view illustrating a display screen according to the present disclosure; and

FIG. 15 is a block diagram illustrating a hardware configuration of a computer according to the present disclosure.

### EXAMPLE EMBODIMENT

### First Example Embodiment

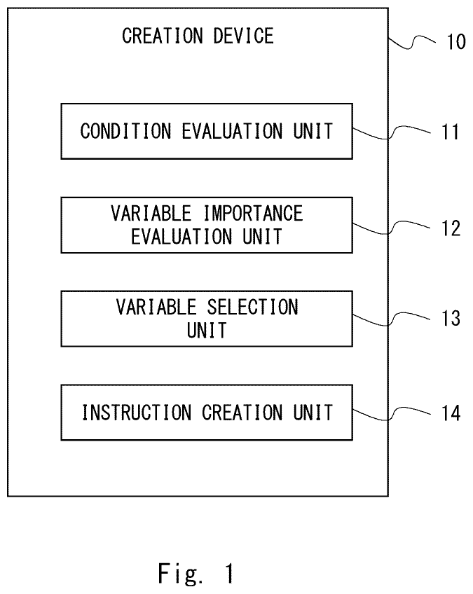

FIG. 1 is a block diagram illustrating a creation device 10 according to the present disclosure. The creation device 10 includes a condition evaluation unit 11, a variable importance evaluation unit 12, a variable selection unit 13, and an instruction creation unit 14. The condition evaluation unit 11, the variable importance evaluation unit 12, the variable selection unit 13, and the instruction creation unit 14 may be software or a module in which processing is executed by a processor executing a program stored in a memory. The condition evaluation unit 11, the variable importance evaluation unit 12, the variable selection unit 13, and the instruction creation unit 14 may be hardware such as a circuit or a chip.

The condition evaluation unit 11 uses the information recorded in the medical/health information database as an input to evaluate a specific health condition of an individual. The variable importance evaluation unit 12 calculates the variable importance obtained by quantifying the magnitude of contribution to the evaluation of the health condition for each variable using information including one or more variables used for the evaluation of the health condition as an input. Using the variable importance as an input, the variable selection unit 13 determines whether to include each variable in the summary sentence. The instruction creation unit 14 creates a summary sentence creation instruction for creating a summary sentence summarizing information regarding the health condition using the variable determined to be included in the summary sentence as an input.

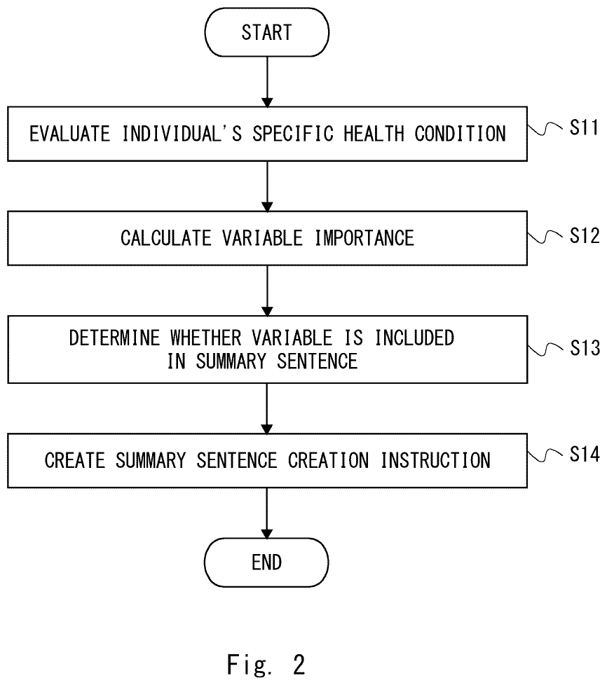

FIG. 2 is a flowchart illustrating a creation method according to the present disclosure. First, the condition evaluation unit 11 evaluates a specific health condition of an individual (step S11). Next, the variable importance evaluation unit 12 calculates the variable importance obtained by quantifying the magnitude of contribution to the evaluation of the health condition for each variable (step S12). Next, the variable selection unit 13 determines whether to include each variable in the summary sentence (step S13). Next, the instruction creation unit 14 creates a summary sentence creation instruction for creating a summary sentence summarizing information regarding the health condition (step S14).

As described above, the creation device 10 calculates the variable importance obtained by quantifying the magnitude of contribution to the evaluation of the health condition of the individual for each variable, and determines whether to include each variable in the summary sentence. As a result, the creation device 10 enables creation of appropriate summary information for an individual.

### Second Example Embodiment

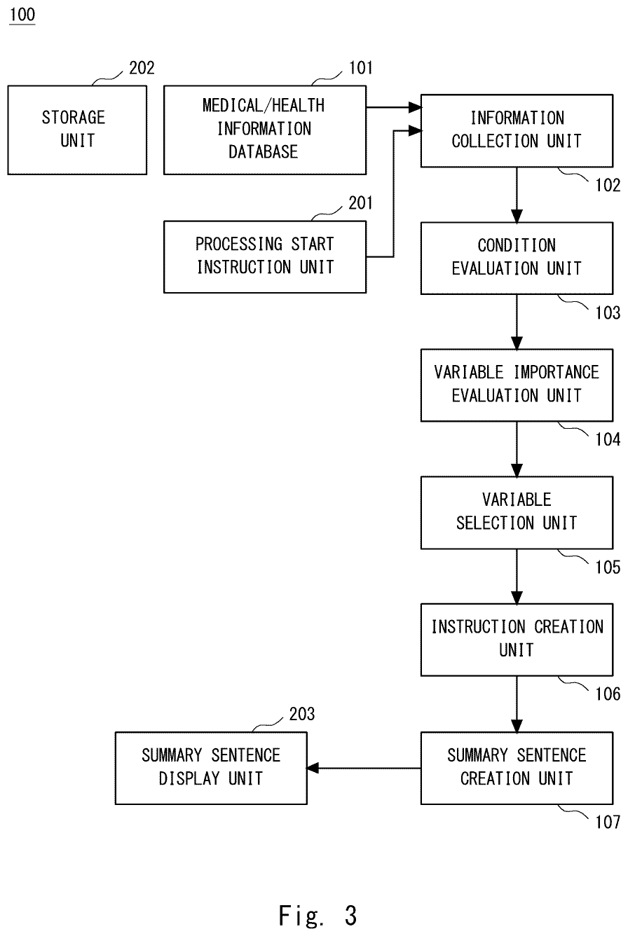

FIG. 3 is a block diagram illustrating a creation device 100 according to the present disclosure. The creation device 100 is a specific example of the creation device 10 described above. The creation device 100 includes a medical/health information database 101, an information collection unit 102, a condition evaluation unit 103, a variable importance evaluation unit 104, a variable selection unit 105, an instruction creation unit 106, and a summary sentence creation unit 107. The creation device 100 includes a processing start instruction unit 201, a storage unit 202, and a summary sentence display unit 203. An example in which an individual to whom the summary sentence is to be prepared is a patient who has been hospitalized for the purpose of surgery will be specifically described.

The medical/health information database 101 is provided in a data warehouse (DWH) of an electronic medical record or a departmental system. The medical/health information database 101 may be an on-premises server installed in a hospital, or may be a database on a cloud service operating on a server installed in another place. The medical/health information database 101 stores information on a target individual. The personal information may include information that is not necessary for evaluating the condition of the hospitalized patient, such as patient number, address, family structure, and primary care physician. The personal information may include a medical record of the individual. The medical record may include static data and time-series data described below. The personal information may be classified in advance into information representing a patient's complaint, objective information such as an inspection value, information representing treatment, and the like.

The information collection unit 102 receives the processing start instruction transmitted from the processing start instruction unit 201. In a case where an input is received from the user through an input unit (not illustrated) or in a case where a predetermined condition is satisfied, the processing start instruction unit 201 transmits a processing start instruction to the information collection unit 102. In a case where the processing start instruction is received, the information collection unit 102 acquires information necessary for condition evaluation of a target patient from the medical/health information database 101 using a key (for example, patient number) that can identify the patient. Then, the information collection unit 102 outputs information necessary for the condition evaluation of the target patient to the condition evaluation unit 103.

The information necessary for the condition evaluation of the patient is used by an algorithm or a machine learning model used by the condition evaluation unit 103. The information necessary for the condition evaluation of the patient varies depending on the algorithm and the machine learning model. The information collection unit 102 collects information necessary for the condition evaluation of the patient based on a predetermined information item, that is, a list of variables.

The collected information includes static data such as basic information of an individual and time-series structured data (also referred to as time-series data) related to a health condition of the individual. The static data represents gender, age, primary disease, comorbidities, medical history, surgical procedure, surgical duration, etc. The time-series data represents vital signs such as a respiratory rate and a heart rate, a state of a surgical wound site, presence or absence, a site, a degree, and a property of pain, a cognitive function, a meal intake amount, results of various specimen inspections, and the like. In a case where the collected information is viewed as variables, these variables reflect the risk of potential complications of the patient and the recovery state after surgery.

The variable name used in the algorithm and the variable name in the medical/health information database 101 may have the same meaning but different form such as “Age” and “Age [y/o]”. In this case, the information collection unit 102 may collect information while referring to the storage unit 202 that stores a conversion table for replacing two variable names.

The condition evaluation unit 103 is a specific example of the condition evaluation unit 11 described above. The condition evaluation unit 103 uses information necessary for condition evaluation of a target patient as an input to evaluate and output a specific health condition of the patient. Here, the specific health condition represents the presence or absence of deviation from the normal progress, the presence or absence of occurrence of complications, the risk of occurrence of future complications, the risk of prolongation of the hospital stay from the scheduled hospital stay, and the like.

The condition evaluation unit 103 evaluates the patient condition from the input information by a predetermined algorithm. This algorithm may use a machine learning method such as a neural network or eXtreme Gradient Boosting, or a language model. In a case where the machine learning method is used, a parameter learned from data of past hospitalized patients in a hospital or the like can be used as a parameter of the machine learning model.

Different algorithms or machine learning models may be used depending on the condition to be evaluated. Information necessary for condition evaluation varies depending on an algorithm or a machine learning model. Therefore, the information collection unit 102 collects different information according to the condition to be evaluated.

The variable importance evaluation unit 104 is a specific example of the variable importance evaluation unit 12. The variable importance evaluation unit 104 calculates the variable importance obtained by quantifying the magnitude of contribution to the evaluation for each variable using the variable used for the evaluation by the condition evaluation unit 103 as an input. For example, the variable importance evaluation unit 104 calculates how much each variable representing the operation time, the results of various specimen inspections, and the like contributes to the evaluation by the condition evaluation unit 103. The variable importance evaluation unit 104 can calculate the variable importance using any known technique.

The variable selection unit 105 is a specific example of the variable selection unit 13. The variable selection unit 105 determines variables to be included in the summary sentence based on the variable importance. For example, the variable selection unit 105 may determine ten variables from a variable having the largest variable importance to a variable having the tenth largest variable importance. For example, the variable selection unit 105 may normalize the variable importance to a range of 0 to 1 and determine a variable in which the value of the normalized variable importance is 0.5 or more. The condition for variable determination may be specified by the user on the user interface.

Different variable importance may be assigned to data at a plurality of time points included in one time-series data. Therefore, data at each time point may or may not be included in the summary sentence. For example, even for the same blood pressure value, the value one week before may be determined to be less important than the most recent value. Depending on the determination method, it may be determined that only the most recent data is included in the summary sentence. The variable importance evaluation unit 104 may calculate the variable importance using data at each of a plurality of time points included in one time-series data as a variable.

The instruction creation unit 106 is a specific example of the instruction creation unit 14. The instruction creation unit 106 creates a summary sentence creation instruction for creating a summary sentence summarizing the health condition of an individual, using the variable determined by the variable selection unit 105 as an input. The summary sentence creation instruction is a prompt for causing a language model such as a GPT (Generative Pretrained Transformer) to create a sentence. The summary sentence creation instruction includes information such as the presence or absence of pain, a change in the region, the degree, and the property, the presence or absence of excretion, and the property of excrement.

The summary sentence creation unit 107 creates a summary sentence using a language model such as GPT with a prompt as an input. The summary sentence creation unit 107 displays the created summary sentence on the summary sentence display unit 203. The summary sentence display unit 203 is a display device such as a display. The language model may be a model specialized in terms related to medical care and healthcare, or may be a general-purpose model. The language model needs to be at least a model relevant to the language desired by the user.

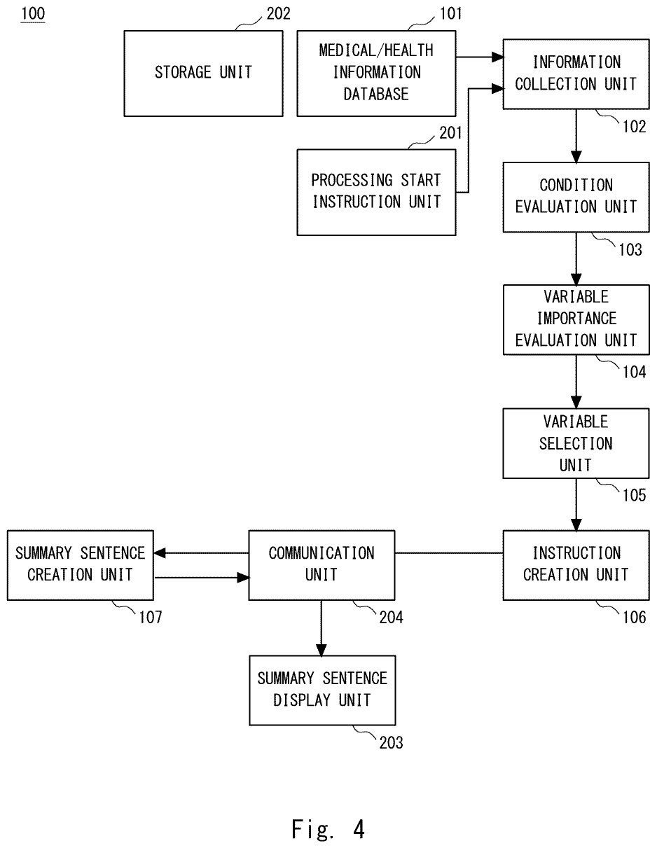

The summary sentence creation unit 107 may operate on the same computer or server as the other constituent elements, or may operate on another computer or server via an application programming interface (API) or a Web API. As illustrated in FIG. 4, the instruction creation unit 106 may transmit a summary sentence creation instruction from a communication unit 204 to the summary sentence creation unit 107 to cause the summary sentence creation unit 107 to create a summary sentence.

As described above, it is possible to create a summary sentence including information regarding a slight change in the health condition and including an element important for the condition evaluation of a specific individual. The medical record information of JP 2020-38602 A does not include information regarding a slight change in health condition, and there is a problem that information regarding a slight change in health condition cannot be reflected in the summary sentence. In a case where the time-series data is input, it is not necessary to newly develop a text analysis unit in a case where the technology is diverted to a different language.

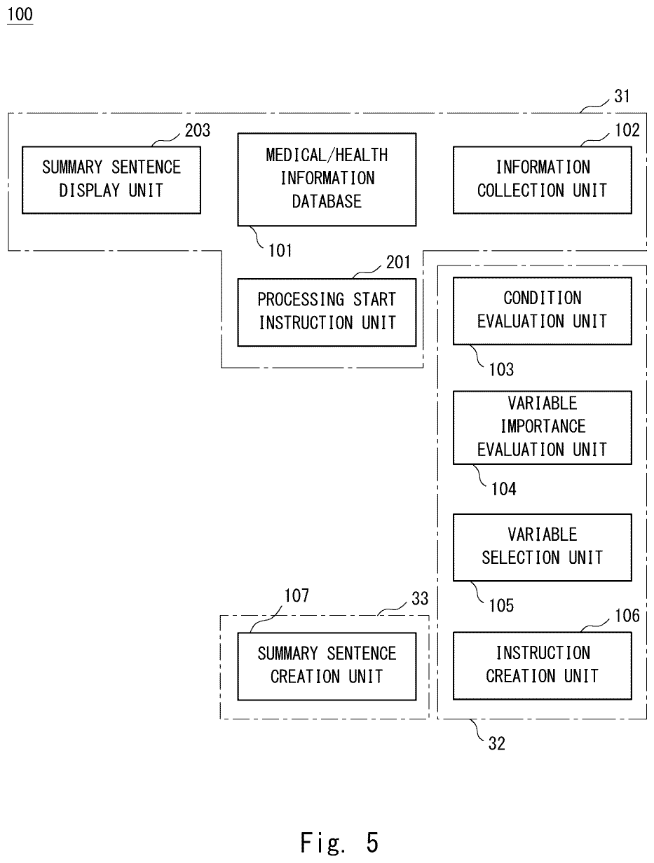

FIG. 5 is a block diagram illustrating a system configuration of the creation device 100. The creation device 100 includes servers 31 to 33. Each of the servers 31 to 33 may include a storage unit (not illustrated). For example, the storage unit 202 provided with a conversion table related to the medical/health information database 101 is provided in the server 31. Each of the servers 31 to 33 may include a communication unit (not illustrated). For example, the communication unit 204 that transmits the summary sentence creation instruction may be provided in the server 32.

The medical/health information database 101, the information collection unit 102, the processing start instruction unit 201, and the summary sentence display unit 203 are provided in the server 31. The server 31 may be, for example, an on-premises server provided in a hospital. The condition evaluation unit 103, the variable importance evaluation unit 104, the variable selection unit 105, and the instruction creation unit 106 are provided in the server 32. The server 32 is an external server provided outside the hospital. The summary sentence creation unit 107 is provided in the server 33. The server 33 is an external server different from the server 32. The system configuration of the creation device 100 is not limited to the configuration illustrated in FIG. 5, and any system configuration can be adopted.

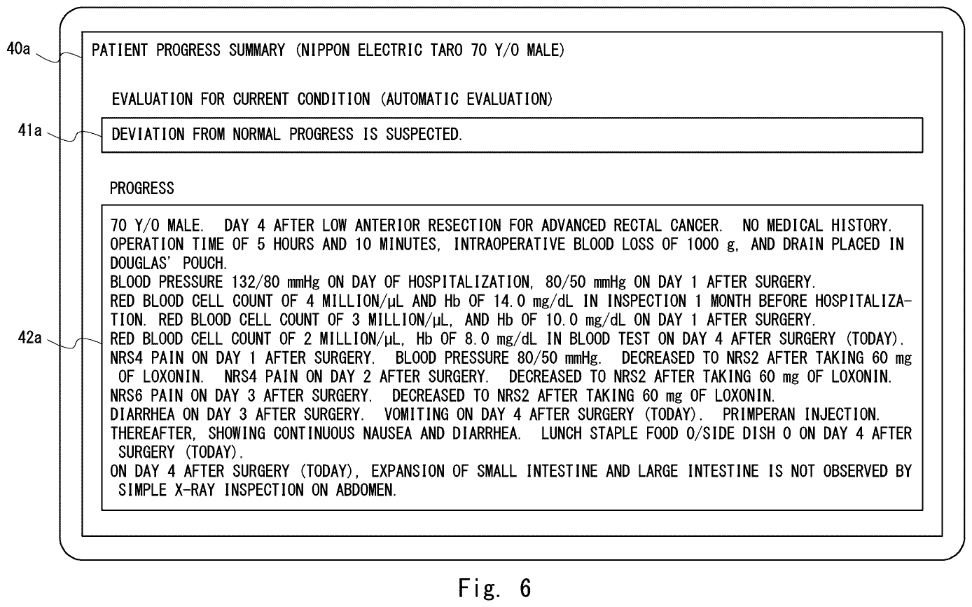

Next, a method for displaying a summary sentence will be described. FIG. 6 is a schematic view illustrating a display screen 40a according to the present disclosure. The display screen 40a includes a display area 41a in which the evaluation for the current condition is displayed and a display area 42a in which a summary sentence of the progress of the patient is displayed. The evaluation for the current condition may be evaluation by the condition evaluation unit 103. The summary sentence includes basic information, preoperative information, surgical information, and postoperative progress. The summary sentence may include all or only some of these pieces of information. The summary sentence of the progress of the patient and the evaluation for the current condition may be displayed in one display area.

The style of the summary sentence may take a form such as itemized writing or sentences. The style of the summary sentence may be in the form of a problem list, which is generally used for describing a medical record, or in the form of SOAP (Subjective information, Objective information, Assessment, Plan). The summary creation instruction may include information specifying a style of the summary sentence.

Whether to include each piece of information in the summary sentence and the style to be selected can be changed according to the attribute of the user or the preference of the user. The attribute of the user represents, for example, an attending physician of a patient, an on-duty physician who is not in charge of the patient, a nurse, or the like. The preference of the user indicates, for example, whether itemized writing in chronological order is good or a sentence is good. For example, in a case where the user is well aware of the progress of the patient, such as a case where the user is the attending physician of the patient, the information included in the summary sentence may be narrowed down to the most recent information among the information regarding the postoperative progress. As a result, the summary sentence can be shortened, and the time required for grasping the summary sentence can be shortened. The above-described user's preference is not limited to whether itemized writing is good or a sentence is good, and the user can create a template of a summary sentence and include the template in the summary creation instruction, thereby creating the summary sentence more flexibly according to the user's preference.

Information such as basic information, preoperative information, surgical information, postoperative progress, and evaluation for current condition may be classified into two categories as normal progress and deviation from normal progress. Among the two categories, only information relevant to a deviation from the normal progress may be included in the summary sentence. Even in a case where the information of the two categories is included in the summary sentence, the information classified into the two categories may be displayed in different modes. The different modes include, for example, different colors of characters for displaying information, and information classified into any category is surrounded by a frame. From the normal progress to the deviation from the normal progress may be classified into a plurality of three or more stepwise categories, and the information classified into each category may be displayed in different modes.

Similarly to the data registered in the medical/health information database 101, the elements included in the summary sentence may be classified into information indicating a patient's complaint, objective information such as an inspection value, and information indicating treatment. Each element is displayed in a different mode according to the classification result. That is, the creation device 100 may display a plurality of elements in the summary sentence in different modes according to the classification of variables serving as sources of the elements in the medical/health information database 101.

A specific past time point or period may be specified, and a summary sentence of the specified time point or period may be created. In this case, it is easy to create a summary sentence for a patient who has been hospitalized for a long time, for each period such as immediately after hospitalization and immediately before discharge. It also facilitates comparing a summary sentence of the most recent progress of a patient with a summary sentence of the past progress of the same or a different patient. It is easy to create announcement materials for case reports.

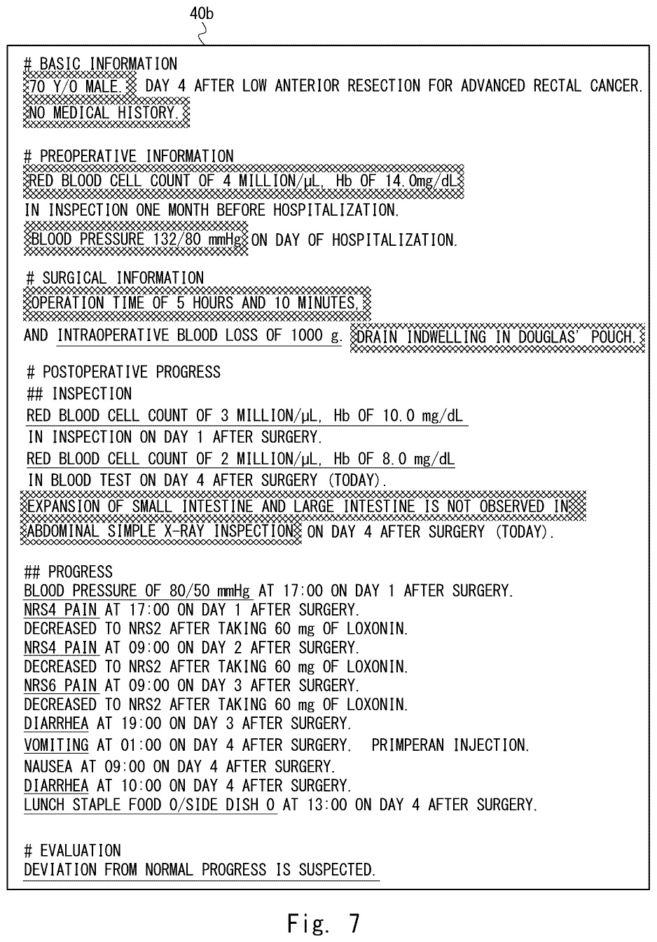

FIG. 7 is a schematic view illustrating a display screen 40b according to the present disclosure. Each of the basic information, the preoperative information, the surgical information, the postoperative progress, and the evaluation for the current condition is itemized in chronological order. In this example, elements indicating a normal progress are shaded, and elements indicating a deviation from the normal progress are highlighted in underline.

For each of the elements indicating the normal progress and the element indicating the deviation from the normal progress, the color of the highlight may be darker as the variable importance calculated by the variable importance evaluation unit 104 is higher. That is, the elements relevant to the variables may be displayed in different modes based on the variable importance.

Different variable importance may be assigned to data at a plurality of time points included in one time-series data. Thus, data at one point in time may be highlighted and data at another point in time may not be highlighted.

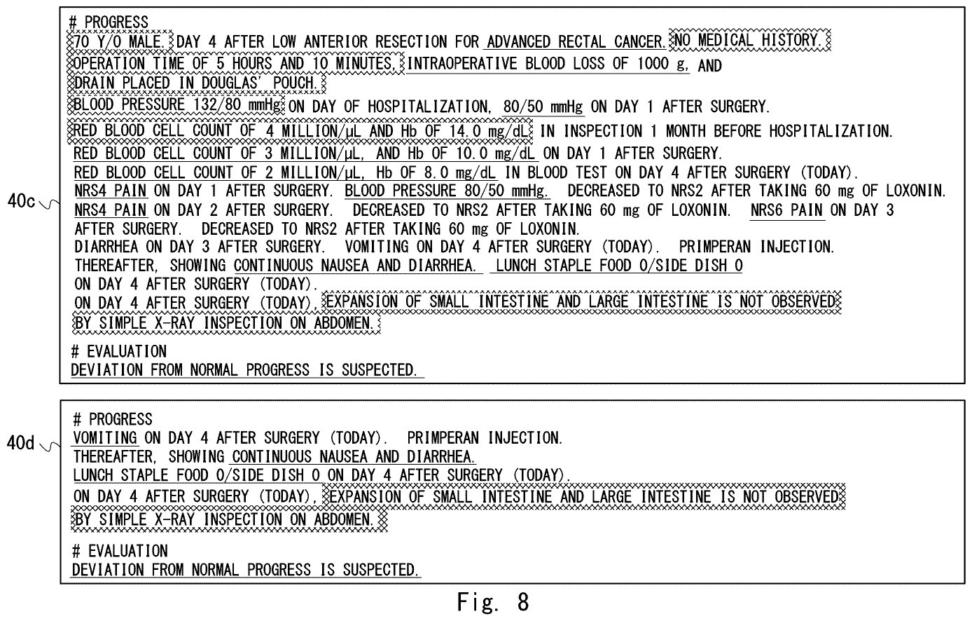

FIG. 8 is a schematic view illustrating display screens 40c and 40d according to the present disclosure. A summary sentence including the latest information and the past information is described on the display screen 40c. Each of the basic information, the preoperative information, the surgical information, the postoperative progress, and the evaluation for the current condition is described as a sentence in chronological order. A summary sentence including only the latest information is described on the display screen 40d. On the display screen 40c, the postoperative progress and the evaluation for the current condition are described as sentences in chronological order. In the display screens 40c and 40d, elements indicating a normal progress are shaded, and elements indicating a deviation from the normal progress are highlighted in underline.

As described above, for each of the elements indicating the normal progress and the element indicating the deviation from the normal progress, the color of the highlight may be darker as the variable importance calculated by the variable importance evaluation unit 104 is higher. That is, the information may be displayed in different modes according to the variable importance.

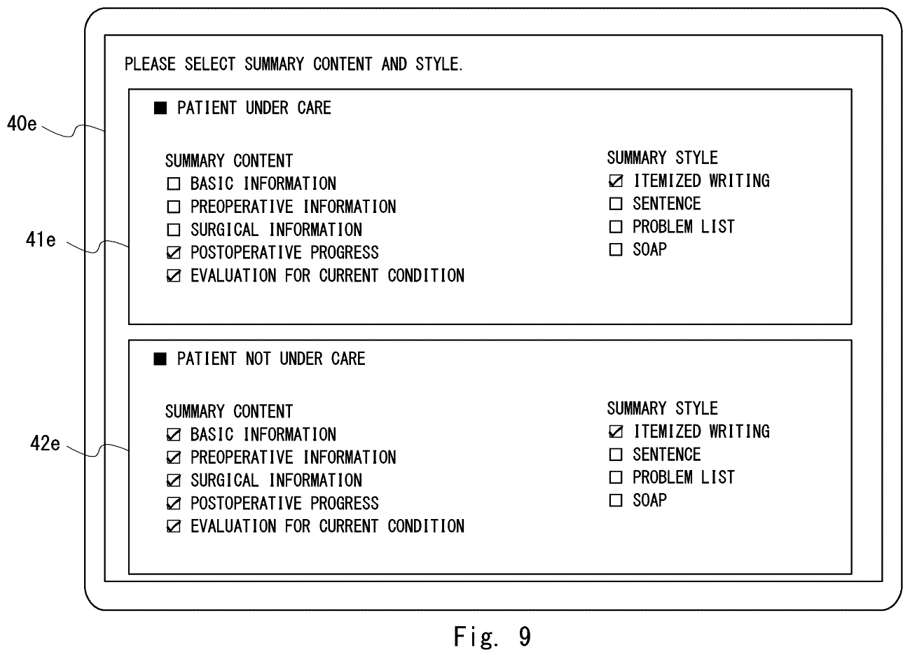

FIG. 9 is a schematic view illustrating a display screen 40e for selecting information included in a summary sentence and a style of the summary sentence. The display screen 40e includes display areas 41e and 42e. The information included in the summary sentence is selected from basic information, preoperative information, surgical information, postoperative progress, and evaluation for the current condition. The style of the summary sentence is selected from itemized writing, sentence format, problem list format, and SOAP format. The user selects the content to be displayed in the summary sentence for the patient under care and the display format of the summary sentence via the display area 41e. The user selects, via the display area 42e, the content to be displayed in the summary sentence for the patient other than the patient under care and the display format of the summary sentence.

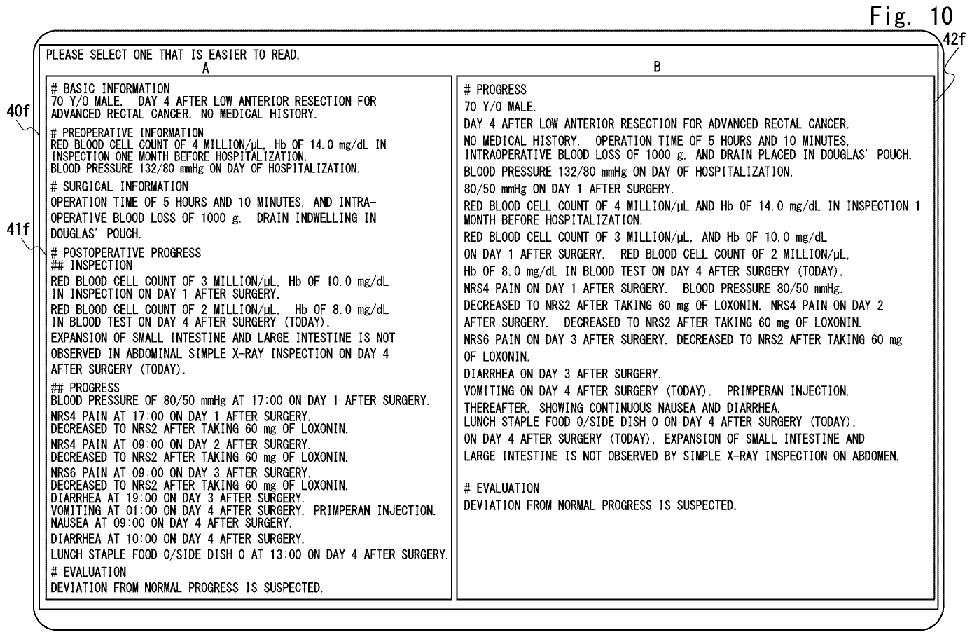

FIG. 10 is a schematic view illustrating a display screen 40f for selecting information included in a summary sentence and a style of the summary sentence. The display screen 40f includes two display areas 41f and 42f. The display screen 40f may include three or more display areas. At least one of the information included in the summary sentence and the style of the summary sentence is different between the display area 41f and the display area 42f. For example, the summary sentence displayed in the display area 41f is described in an itemized form, and the summary sentence displayed in the display area 42f is described in a sentence form. The user can set the information included in the summary sentence and the style of the summary sentence by inputting an operation of selecting one of the display areas 42e and 42f.

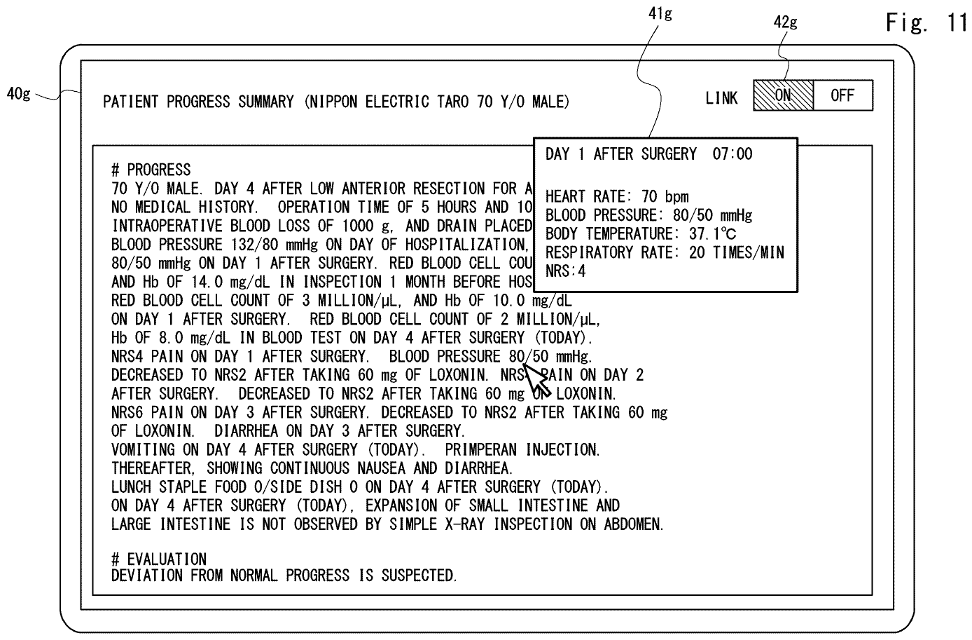

FIG. 11 is a schematic view illustrating a display screen 40g according to the present disclosure. The display screen 40g includes a window 41g and a button 42g. The summary sentence displayed on the display screen 40g has a link to an inspection value and a sentence which are the basis of the summary sentence. In a case where the linked portion is clicked, the linked information (for example, the inspection value) is displayed in the window 41g. In a case where the window 41g is displayed, it may be difficult to read the summary sentence. Therefore, the button 42g for switching between display and non-display of the window 41g is provided. The linked information can be displayed without using the window 41g. In a case where a second pane different from the first pane in which the summary sentence is displayed is fixedly displayed, the linked information may be configured to be displayed in the second pane.

There is a known problem that a language model may output a sentence including information different from the fact. The presence of the inspection value and the link to the sentence as the basis of the summary sentence allows the user to confirm that the information included in the summary sentence is correct information.

The processing start instruction unit 201 will be described in detail with reference to FIG. 3 again. The processing start instruction unit 201 may receive an input from the user, may give a processing start instruction at regular time intervals, or may give a processing start instruction according to the patient condition evaluated by the condition evaluation unit 103.

In a case where the input from the user is received, for example, a summary creation button may be provided on a user interface for inputting information such as an electronic medical record to the medical/health information database 101 and referring to the information such as the electronic medical record. The user instructs to create a summary sentence via the summary creation button. The creation device 100 creates a summary sentence creation instruction or creates a summary sentence, and displays the summary sentence on the user interface. As a result, the creation device 100 can support the medical staff who has visited the hospital room to grasp information by rounding or notification of a medical nurse call. In particular, the creation device 100 can support a medical staff who has not been involved with the patient to immediately grasp the progress of the patient in a case where the condition of the patient suddenly changes. This method may not receive an explicit instruction from the user. For example, in a case where the user inputs a predetermined information item into the electronic medical record, it is determined whether the information satisfies a predetermined condition, and in a case where the condition is satisfied, subsequent processing can be executed. For example, in a case where a value in which pain felt by a patient is answered in 11 stages from 0 to 10 is input to an electronic medical record or the like, if the value is a predetermined value or more, processing of generating and displaying a summary is executed. The predetermined value may be determined for each information item by each user, or may be determined for each information item by an administrator or the like of the electronic medical record as a rule of a facility such as a hospital. According to this method, even in a case where the user is not suspected of an abnormality of the patient condition, evaluation of the patient condition, generation of a summary, and display processing are automatically executed, and an effect of making the user aware of the abnormality of the patient condition is generated.

In the method of giving a processing start instruction at regular time intervals, for example, the creation device 100 simultaneously creates a summary sentence for each patient meeting a specific condition among the patients registered in the medical/health information database 101. The specific condition may include that the patient is currently hospitalized, that the patient is currently hospitalized and the evaluation by the condition evaluation unit 103 is worse than a certain standard, that the patient is a patient designated by the user, or the like.

For example, in a case where the specific condition is set as being any hospitalized patient in a certain ward, and the processing start instruction by the processing start instruction unit 201 is automatically output at 7:00 every morning, a summary sentence is created before the start of work in the daily shift for each patient hospitalized in the ward. Therefore, the creation device 100 can facilitate the doctor and the nurse to grasp the progress of all the patients under care at the time of coming to work. The creation device 100 can output the evaluation by the condition evaluation unit 103 at the same time as the summary sentence, and can support preferentially checking the progress of a patient with a particularly poor condition among patients who are hospitalized in the ward.

In the method of starting the processing according to the patient's condition evaluated by the condition evaluation unit 103, the condition evaluation unit 103 performs the condition evaluation every fixed time or every time new data is input to the medical/health information database 101. In a case where the evaluated condition is inferior to a certain standard, the creation device 100 creates a summary sentence creation instruction or creates a summary sentence.

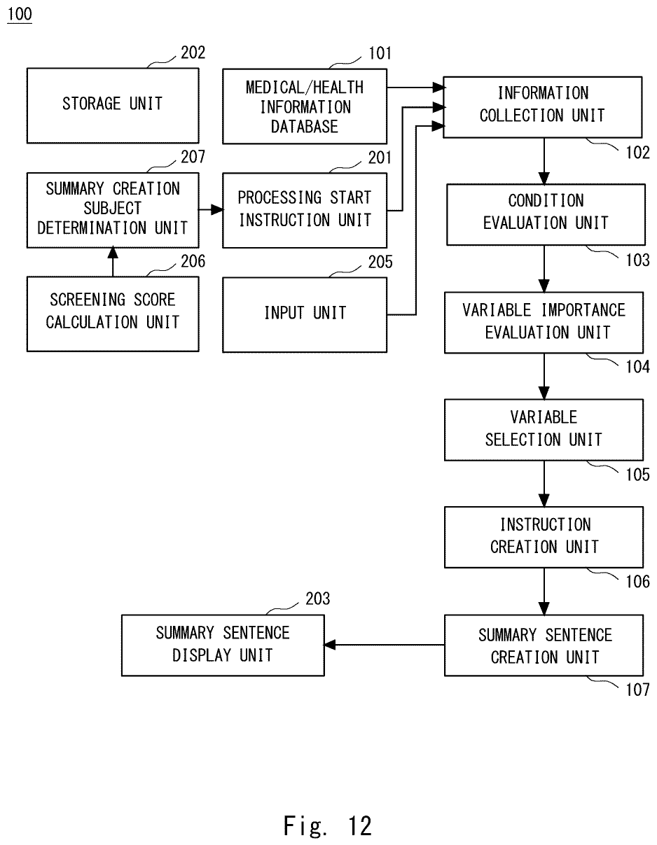

The creation device 100 may extract a patient using a scoring system different from the condition evaluation unit 103 and evaluate the condition of the extracted patient. FIG. 12 is a block diagram illustrating the creation device 100 according to the present disclosure. Comparing FIG. 3 with FIG. 12, the creation device 100 illustrated in FIG. 12 includes an input unit 205, a screening score calculation unit 206, and a summary creation subject determination unit 207. As described above, the instruction to start the processing may be issued via the input unit 205. The input unit 205 may receive an operation of selecting information included in the summary sentence or a format of the summary sentence.

The scoring system is, for example, an early warning scoring system (EWSS) used in a rapid response system (RRS). The summary creation subject determination unit 207 determines a patient who is a target for creating a summary sentence based on the score calculated by the screening score calculation unit 206. In the case of using the rapid response system, the medical staff can grasp the past progress of the patient in a shorter time.

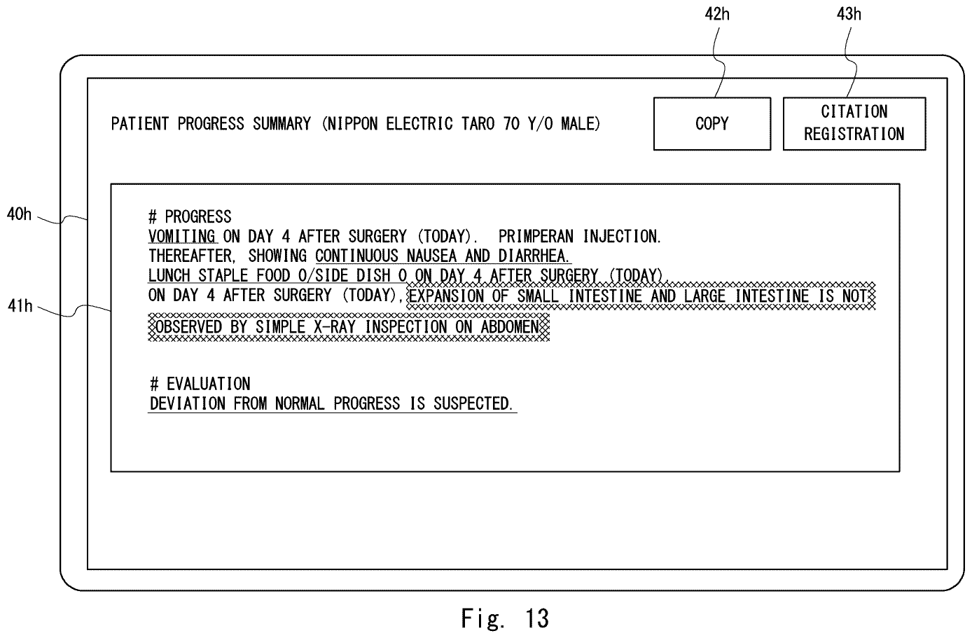

Next, a method of registering the created summary sentence as a record by citing the created summary sentence will be described. FIG. 13 is a schematic view illustrating a display screen 40h of a summary sentence. The display screen 40h includes a display area 41h in which a summary sentence is displayed, a button 42h, and a button 43h. The user can select the button 42h, copy the contents of the summary sentence, and paste the contents into a form for registering information. In a case where the user selects the button 43h, the creation device 100 causes the display screen 40h to transition to an input screen for various kinds of recording. An input screen in a state where the summary sentence is input to the input form may be displayed. Citing the summary sentence can shorten the time for the medical staff to create a record.

In a case where a summary sentence is created in a case where an input from a user is received, in a case where a summary sentence is created based on a result evaluated by the condition evaluation unit 103, or in a case where a summary sentence is created for a patient extracted using a scoring system different from the condition evaluation unit 103, it is considered that the risk and interest for the patient are higher as a summary sentence for a certain patient is created more frequently. Therefore, the generation frequency of the summary sentence can be regarded as an index of the condition of the patient in the entire hospital. The creation device 100 can also create a dashboard for grasping the condition of the patient in the entire hospital. By unitarily grasping the state of the entire hospital, it is possible to ensure patient safety and efficiently allocate personnel.

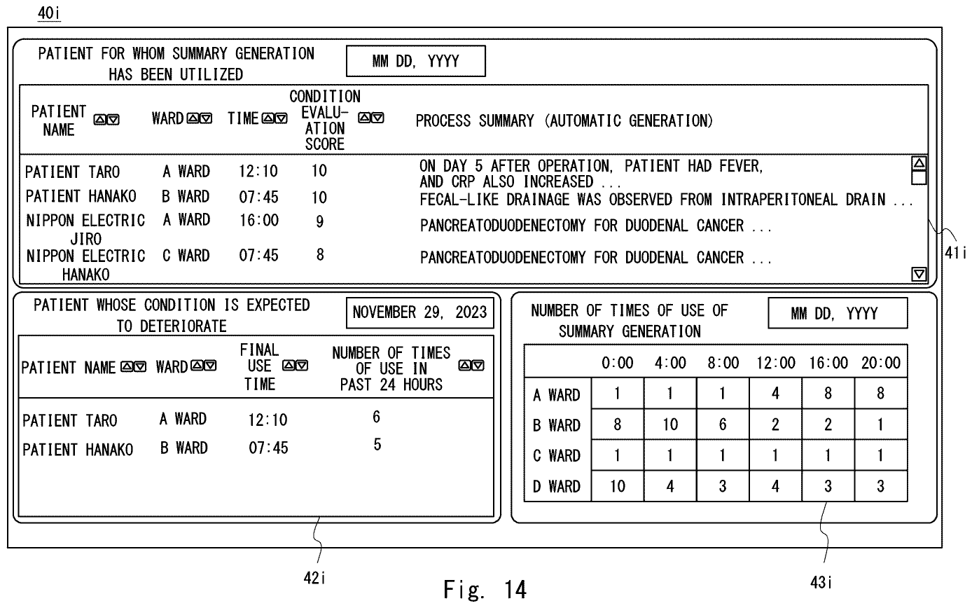

FIG. 14 is a schematic view illustrating a display screen 40i of a dashboard. The display screen 40i includes a patient list 41i, a table 42i, and a table 43i. The patient list 41i includes the patient name of each patient, the ward, the time in a case where the summary sentence has been created, the evaluation score by the condition evaluation unit 103, and the summary sentence. The table 42i shows the number of times a summary sentence has been prepared for each patient in a predetermined time in the past. The table 43i shows the number of times a summary sentence has been created for a patient hospitalized in each ward for each time zone. By checking the table 43i, it is possible to grasp the busyness of the medical staff in each ward. Although not illustrated, it is also possible to display, on the display screen 40i of the dashboard, indices relating to patient safety and the busy degree of the ward other than input and output of the creation device 100. The indices are, for example, the number of hospitalized patients, the hospital bed utilization rate, the necessity of nursing, and the like for each ward.

According to the second example embodiment, it is possible to shorten the time taken for a medical and health care expert to search for information from the medical/health information database 101 and to prevent overlooking of the information. It is possible to shorten a time for summarizing information in a case where an expert (for example, a nurse) shares or discusses information with another expert (for example, other nurses, doctors). In addition to the electronic medical record, the second example embodiment can also target a personal health record (PHR) collected by a health app or the like.

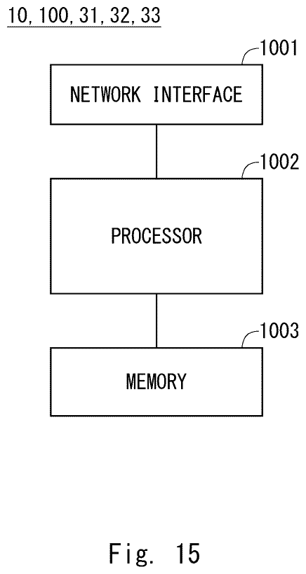

FIG. 15 is a block diagram illustrating a hardware configuration of the creation device 10, the creation device 100, the server 31, the server 32, and the server 33 (hereinafter, referred to as the creation device 10 or the like). Referring to FIG. 15, the creation device 10 and the like include a network interface 1001, a processor 1002, and a memory 1003. The network interface 1001 is used to communicate with other network node apparatuses that constitute a communication system.

The processor 1002 reads and executes software (computer program) from the memory 1003 to perform the processing in Step S11 to S14 in FIG. 2. The processor 1002 may be, for example, a microprocessor, an MPU, or a CPU. The processor 1002 may include a plurality of processors.

The memory 1003 is constituted by a combination of a volatile memory and a nonvolatile memory. The memory 1003 may include a storage disposed away from the processor 1002. In this case, the processor 1002 may access the memory 1003 via an input/output (I/O) interface, which is not illustrated.

In the example in FIG. 15, the memory 1003 is used to store software modules. The processor 1002 can perform the processing in steps S11 to S14 by reading and executing the software modules from the memory 1003.

As described with reference to FIG. 15, each of the processors included in the creation device 10 and the like in the above-described example embodiments executes one or a plurality of programs including instructions for causing a computer to perform the algorithm described with reference to the drawings.

In the above-described example, the program includes instructions (or software codes) for causing the computer to perform one or more functions described in the example embodiments, in a case of being read by the computer. The program may be retained in a non-transitory computer-readable medium or a tangible storage medium. As an example and not by way of limitation, the computer-readable medium or the tangible storage medium includes a random-access memory (RAM), a read-only memory (ROM), a flash memory, a solid-state drive (SSD) or any other memory technique, a CD-ROM, a digital versatile disc (DVD), a Blu-ray (registered trademark) disc or any other optical disc storage, and a magnetic cassette, a magnetic tape, a magnetic disk storage, or any other magnetic storage device. The programs may be transmitted through a transitory computer-readable medium or a communication medium. As an example and not by way of limitation, transitory computer-readable media or communication media include electrical, optical, acoustic, or other forms of propagated signals.

The technical ideas of the present disclosure are not limited to the above example embodiment and can be appropriately modified without departing from the scope.

While the present disclosure has been particularly shown and described with reference to example embodiments thereof, the present disclosure is not limited to these example embodiments. It will be understood by those of ordinary skill in the art that various changes in form and details may be made therein without departing from the spirit and scope of the present disclosure as defined by the claims. And each example embodiment can be appropriately combined with other example embodiments.

Each of the drawings is merely illustrative for describing one or more example embodiments. Each of the drawings is not associated with only one specific example embodiment, but may be associated with one or more other example embodiments. As those of ordinary skill in the art will appreciate, various features or steps described with reference to any one of the drawings may be combined with features or steps illustrated in one or more other drawings, for example, to create an example embodiment not explicitly illustrated or described. All of the features or steps illustrated in any one of the drawings for explaining illustrative example embodiments are not necessarily mandatory, and some features or steps may be omitted. The order of the steps illustrated in any of the drawings may be changed as appropriate.

Some or all of the above-described example embodiments may be described as the following Supplementary Notes, but are not limited to the following.

Some or all of the elements (e.g., configurations and functions) described in Supplementary Notes 2 to 11 subordinate to Supplementary Note 1 may also be subordinate to Supplementary Notes 12 and 13 due to the subordinate relationships similar to those of Supplementary Notes 2 to 11. Some or all of the elements described in any supplementary note may be applied to various types of hardware, software, recording means for recording software, systems, and methods.

### Supplementary Note 1

A creation device including:
a condition evaluation unit that evaluates a specific health condition of an individual using information recorded in a medical/health information database as an input;
a variable importance evaluation unit that calculates a variable importance obtained by quantifying a magnitude of contribution to evaluation of the health condition for each variable using information including one or more variables used for the evaluation of the health condition as an input;
a variable selection unit that determines whether each variable is included in a summary sentence using the variable importance as an input; and
an instruction creation unit that creates a summary sentence creation instruction for creating a summary sentence summarizing information regarding the health condition using a variable determined to be included in the summary sentence as an input.

### Supplementary Note 2

The creation device according to Supplementary Note 1, including a summary sentence creation unit that creates the summary sentence using the summary sentence creation instruction as an input.

### Supplementary Note 3

The creation device according to Supplementary Note 2, in which the condition evaluation unit evaluates the health condition using at least time-series structured data among the information recorded in the medical/health information database as an input.

### Supplementary Note 4

The creation device according to Supplementary Note 2, in which the condition evaluation unit evaluates the health condition using at least sentence data among the information recorded in the medical/health information database as an input.

### Supplementary Note 5

The creation device according to any one of Supplementary Notes 2 to 4, including a display unit that displays the summary sentence.

### Supplementary Note 6

The creation device according to any one of Supplementary Notes 2 to 4, in which a plurality of elements in the summary sentence are displayed in different modes according to whether a variable relevant to each element indicates a normal progress or a state deviating from a normal progress.

### Supplementary Note 7

The creation device according to any one of Supplementary Notes 2 to 4, in which a plurality of elements in the summary sentence are displayed in different modes according to classifications of variables serving as sources of the elements in the medical/health information database.

### Supplementary Note 8

The creation device according to any one of Supplementary Notes 2 to 4, in which the instruction creation unit creates a summary sentence creation instruction to instruct to write the summary sentence into at least a sentence format and a format other than the sentence format.

### Supplementary Note 9

The creation device according to any one of Supplementary Notes 2 to 4, in which the instruction creation unit creates a summary sentence creation instruction to instruct to write the summary sentence into at least itemized writing and a format other than the itemized writing.

### Supplementary Note 10

The creation device according to Supplementary Note 8 or 9, including an input unit that receives an input of a user specifying a format of the summary sentence.

### Supplementary Note 11

The creation device according to any one of Supplementary Notes 2 to 4, further including:
a display unit that displays two or more summary sentences having different formats; and
an input unit that receives an input of a user selecting any one of the displayed two or more summary sentences.

### Supplementary Note 12

A creation method including:
evaluating a specific health condition of an individual using information recorded in a medical/health information database as an input;
calculating a variable importance obtained by quantifying a magnitude of contribution to evaluation of the health condition for each variable using information including one or more variables used for the evaluation of the health condition as an input;
determining whether each variable is included in a summary sentence using the variable importance as an input; and
creating a summary sentence creation instruction for creating a summary sentence summarizing information regarding the health condition using a variable determined to be included in the summary sentence as an input.

### Supplementary Note 13

A program for causing a computer to execute:
a process of evaluating a specific health condition of an individual using information recorded in a medical/health information database as an input;
a process of calculating a variable importance obtained by quantifying a magnitude of contribution to evaluation of the health condition for each variable using information including one or more variables used for the evaluation of the health condition as an input;
a process of determining whether each variable is included in a summary sentence using the variable importance as an input; and
a process of creating a summary sentence creation instruction for creating a summary sentence summarizing information regarding the health condition using a variable determined to be included in the summary sentence as an input.

1. A creation device comprising:
at least one memory storing instructions and
at least one processor configured to execute the instructions to:
evaluate a specific health condition of an individual using information recorded in a medical/health information database as an input;
calculate a variable importance obtained by quantifying a magnitude of contribution to evaluation of the health condition for each variable using information including one or more variables used for the evaluation of the health condition as an input;
determine whether each variable is included in a summary sentence using the variable importance as an input; and
create a summary sentence creation instruction for creating a summary sentence summarizing information regarding the health condition using a variable determined to be included in the summary sentence as an input.

2. The creation device according to claim 1, wherein the at least one processor is further configured to execute the instructions to:
create the summary sentence using the summary sentence creation instruction as an input.

3. The creation device according to claim 2, wherein the at least one processor is further configured to execute the instructions to:
evaluate the health condition using at least time-series structured data among the information recorded in the medical/health information database as an input.

4. The creation device according to claim 2, wherein the at least one processor is further configured to execute the instructions to:
evaluate the health condition using at least sentence data among the information recorded in the medical/health information database as an input.

5. The creation device according to claim 2, comprising a display that displays the summary sentence.

6. The creation device according to claim 2, wherein a plurality of elements in the summary sentence are displayed in different modes according to whether a variable relevant to each element indicates a normal progress or a state deviating from the normal progress.

7. The creation device according to claim 2, wherein a plurality of elements in the summary sentence are displayed in different modes according to classifications of variables serving as sources of the elements in the medical/health information database.

8. The creation device according to claim 2, wherein the at least one processor is further configured to execute the instructions to:
create a summary sentence creation instruction to instruct to write the summary sentence into at least a sentence format and a format other than the sentence format.

9. A creation method comprising:
evaluating a specific health condition of an individual using information recorded in a medical/health information database as an input;
calculating a variable importance obtained by quantifying a magnitude of contribution to evaluation of the health condition for each variable using information including one or more variables used for the evaluation of the health condition as an input;
determining whether each variable is included in a summary sentence using the variable importance as an input; and
creating a summary sentence creation instruction for creating a summary sentence summarizing information regarding the health condition using a variable determined to be included in the summary sentence as an input.

10. A non-transitory computer-readable medium storing a program for causing a computer to execute:
a process of evaluating a specific health condition of an individual using information recorded in a medical/health information database as an input;
a process of calculating a variable importance obtained by quantifying a magnitude of contribution to evaluation of the health condition for each variable using information including one or more variables used for the evaluation of the health condition as an input;
a process of determining whether each variable is included in a summary sentence using the variable importance as an input; and
a process of creating a summary sentence creation instruction for creating a summary sentence summarizing information regarding the health condition using a variable determined to be included in the summary sentence as an input.
